(19) United States
(12) Patent Application Publication
(10) Pub. No.: US 2022/0249092 A1
(43) Pub. Date: Aug. 11, 2022

(54) SURGICAL TOOL CLAMPING FEEDBACK DEVICE AND METHOD THEREOF

(71) Applicant: Qisda Corporation, Taoyuan City (TW)

(72) Inventor: Zhe-Wei SU, Taoyuan City (TW)

(73) Assignee: Qisda Corporation, Taoyuan City (TW)

(21) Appl. No.: 17/585,669

(22) Filed: Jan. 27, 2022

(30) Foreign Application Priority Data
Feb. 8, 2021 (CN) 202110184168.2

(51) Int. Cl. A61B 17/10; A61B 17/32

(52) U.S. Cl. CPC A61B 17/10; A61B 17/320068; A61B 2017/00022; A61B 2017/320078; A61B 2017/320082; A61B 17/320016

(57) ABSTRACT

A surgical tool clamping feedback device includes a surgical tool, a force feedback sensor and a feedback processing unit. The surgical tool includes a machine body, a harmonic scalpel, a clamp and an operating handle. The machine body is configured to install the harmonic tool head. The harmonic scalpel is configured to install the clamp and output an ultrasonic wave to the clamp. The operating handle is movably disposed on the machine body for a user to hold and operate the clamp. The force feedback sensor is linked with the operating handle to detect a gripping feedback force from the operating handle. The feedback processing unit is configured to receive the grip feedback force of the operating handle and correspondingly output an indication signal to the user.

This application claims the benefit of People's Republic of China application Serial No. 202110184168.2, filed Feb. 8, 2021, the subject matter of which is incorporated herein by reference.

### BACKGROUND OF THE INVENTION

### Field of the Invention

The invention relates in general to a surgical treatment device, and more particularly to a surgical tool clamping feedback device and a method thereof.

### Description of the Related Art

Minimally invasive surgery is a treatment method by cutting a smaller incision and inserting a surgical tool through the incision. Compared with traditional open surgery with a larger incision, the minimally invasive surgery has a smaller incision, which can reduce postoperative recovery time, reduce pains and scars, so that the minimally invasive surgery is widely used.

The effect of surgical tools on organs and tissues can be clearly seen in traditional open surgery, however, minimally invasive surgery has many limitations in term of vision and tactile aspects. Due to the lack of direct tactile feedback, surgeons are not easily aware of the effects of surgical tools on organs and tissues, and cannot determine whether the force or energy applied to the organs and tissues is appropriate, and thus often needs to rely on experience and endoscopy to determine whether an organ or tissue is intact or damaged. Therefore, minimally invasive surgery still has many difficulties for surgeons to overcome.

### SUMMARY OF THE INVENTION

The present invention relates to a surgical tool clamping feedback device and a method thereof, which can provide a clamping feedback force of a clamp or a grip feedback force of an operating handle when a user performs a surgical operation to respond to the user's operation.

According to an aspect of the present invention, a surgical tool clamping feedback device is provided, which includes a surgical tool, a force feedback sensor and a feedback processing unit. The surgical tool includes a machine body, a harmonic scalpel, a clamp and an operating handle, wherein the machine body is configured to install the harmonic scalpel, and the harmonic scalpel is configured to install the clamp and output an ultrasonic wave to the clamp, and the operating handle is movably arranged on the machine body for a user to hold and operate the clamp. The force feedback sensor is linked with the operating handle to detect a grip feedback force of the operating handle. The feedback processing unit is configured to receive the grip feedback force of the operating handle and correspondingly output an instruction signal to the user.

According to an aspect of the present invention, a clamping feedback method of a surgical tool is provided for a surgical operation. The surgical tool includes a machine body, a harmonic scalpel, a clamp and an operating handle, wherein the machine body is configured to install the harmonic scalpel, the harmonic scalpel is configured to install the clamp and output an ultrasonic wave to the clamp. The operating handle is movably disposed on the machine body for a user to hold and operate the clamp. The clamping feedback method of the surgical tool includes the following steps. A force is applied to the operating handle. A grip feedback force of the operating handle is detected. According to the grip feedback force of the operating handle, an instruction signal is correspondingly output to the user.

Other objects, features, and advantages of the invention will become apparent from the following detailed description of the preferred but non-limiting embodiments. The following description is made with reference to the accompanying drawings.

### BRIEF DESCRIPTION OF THE DRAWINGS

FIG. 1 is a schematic diagram of appearance of a surgical tool clamping feedback device according to an embodiment of the present invention.

FIG. 2A is an internal schematic diagram of a tool head according to an embodiment of the present invention.

FIG. 2B is an internal schematic diagram of a tool head according to another embodiment of the present invention.

FIG. 3A is an internal schematic diagram of a surgical tool clamping feedback device according to an embodiment of the present invention.

FIG. 3B is an internal schematic diagram of a surgical tool clamping feedback device according to another embodiment of the present invention.

FIG. 4 is a schematic flow chart of a surgical tool clamping feedback method according to an embodiment of the present invention.

### DETAILED DESCRIPTION OF THE INVENTION

Exemplary embodiments will now be described more fully with reference to the accompanying drawings. However, the exemplary embodiments could be implemented in various forms, and should not be construed as being limited to the examples set forth herein; on the contrary, the description of these embodiments makes the present disclosure more comprehensive and complete, and fully conveys the concept of the exemplary embodiments to those skilled in the art. The described features, structures or characteristics could be combined in one or more embodiments in any suitable way. The same or similar elements will be represented by the same or similar reference signs, and the description will not be repeated.

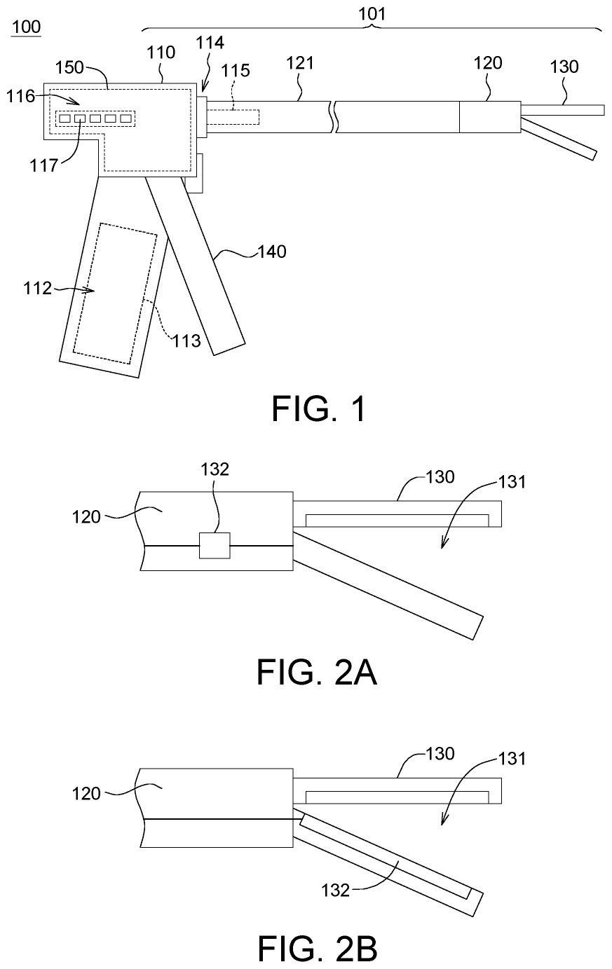

FIG. 1 is a schematic diagram of appearance of a surgical tool clamping feedback device 100 according to an embodiment of the present invention. The surgical tool 101 includes a machine body 110, a harmonic scalpel 120, a clamp 130 and an operating handle 140. The machine body 110 is configured to install the harmonic scalpel 120, and the harmonic scalpel 120 is configured to install the clamp 130 and output an ultrasonic wave to the clamp 130. The operating handle 140 is movably disposed on the machine body 110 for a user to hold and operate the clamp 130.

The surgical tool 101 is, for example, a tool used in laparoscopic surgery, including electrosurgical scissors, a tissue holder, and a dissector.

The laparoscopic lens can be inserted into the abdominal cavity, so that the captured images can be transmitted to the signal processing system through the fiber optic tube and is displayed on the display screen in real time. The harmonic scalpel 120 is also known as an ultrasonic knife. The scalpel can be mechanically oscillated at an ultrasonic frequency through an ultrasonic frequency generator, so that water molecules and protein hydrogen bonds in the tissue can be broken, cells can be disintegrated, and the tissue can be cut or coagulation by the scalpel, and the vessel is closed by clamping the vessel with the clamp 130. The damage of the harmonic scalpel 120 to the surrounding tissue is much smaller than that of the electrosurgical knife. The harmonic scalpel 120 can control the cutting and hemostasis more accurately, can assist in the cutting and separation operation near important organs and large blood vessels, and will not generate smoke to affect the surgical field. Therefore, the laparoscopic surgery has a clear view and the surgery operation time is shortened.

Please refer to FIG. 1, the machine body 110 includes a handle portion 112, a mounting portion 114 and a light signal display portion 116, and a sleeve 115 is disposed in front of the mounting portion 114 of the machine body 110 for mounting the harmonic scalpel 120 and the extension rod 121 thereof. In addition, the handle portion 112 of the machine body 110 is located on a bottom of the mounting portion 114, and the handle portion 112 is configured to operate in cooperation with the operating handle 140 for the user to hold. The operating handle 140 can be pivotally disposed below the mounting portion 114 or on the handle portion 112. The handle portion 112 is detachably disposed on the mounting portion 114, so that the handle portion 112 and the operating handle 140 can be separated from the mounting portion 114. In addition, the handle portion 112 of the machine body 110 has an accommodating space 113 inside, which can be configured to install a battery or other devices (e.g., a force feedback sensor). A display device (not shown) can also be installed on the handle portion 112 of the machine body 110 for displaying the image captured by the camera inside the incision. In addition, a signal display portion 116 of the machine body 110 is located on a rear of the mounting portion 114, and can output an indication signal correspondingly for the user to see. The indication signal is, for example, represented by the LEDs 117 or audio. The LEDs 117 can display lights of different colors or a plurality of continuous LED lights, and the audio signal can be a high-frequency signal or a low-frequency signal from different audio ranges or different audio sources.

Please refer to FIGS. 2A and 2B, which are internal schematic diagrams of a surgical tool clamping feedback device 100 according to an embodiment of the present invention. The force feedback sensor 132 is disposed in the harmonic scalpel 120 or in a jaw 131 of the clamp 130, and the force feedback sensor 132 can be linked with the clamp 130 to detect a clamping feedback force of the clamp 130. The force feedback sensor 132 is, for example, a pressure sensor, a tension sensor, a displacement sensor, an impedance sensor or other sensors that can detect the clamping force. In this embodiment, as long as the clamp 130 clamps the tissue or blood vessel, the force feedback sensor 132 can directly reflect the clamping force of the clamp 130 to respond to the user's operation.

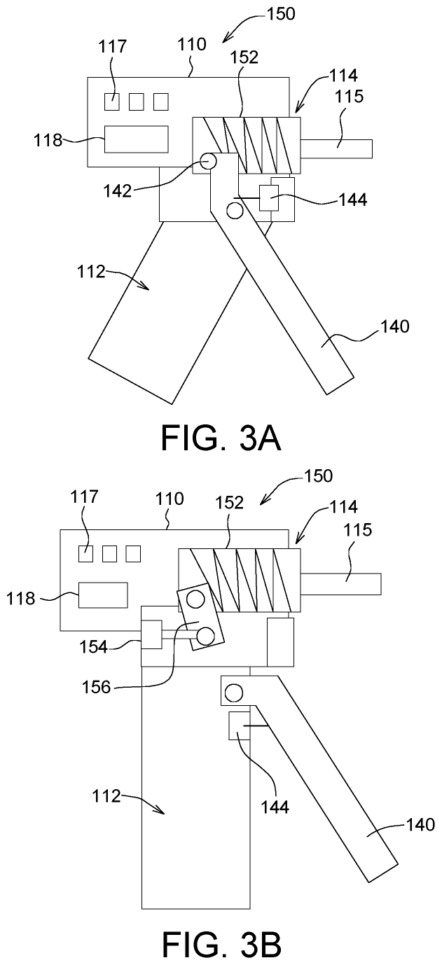

Please refer to FIGS. 3A and 3B, which are internal schematic diagrams of a surgical tool clamping feedback device 100 according to another embodiment of the present invention. In another embodiment, the force feedback sensor 144 can also be disposed in the machine body 110 and linked with the operating handle 140 to detect a grip feedback force of the operating handle 140. The force feedback sensor 144 is, for example, a pressure sensor, a tension sensor, a displacement sensor, an impedance sensor, or other sensors that can detect the grip force of the operating handle 140. In this embodiment, as long as the clamp 130 clamps the tissue or blood vessel, the force feedback sensor 144 can directly reflect the grip force of the operating handle 140, and the grip force of the operating handle 140 is substantially proportional to the clamping force of the clamp 130 to respond to the user's operation. In FIG. 3A, the operating handle 140 and the force feedback sensor 144 may be disposed below the mounting portion 114 of the machine body 110. In FIG. 3B, the operating handle 140 and the force feedback sensor 144 may be disposed on the handle portion 112 of the machine body 110.

Please refer to FIGS. 1, 3A and 3B, the feedback processing unit 150 is disposed in the machine body 110 for receiving the clamping feedback force of the clamp 130 or the grip feedback force of the operating handle 140 to output an indication signal correspondingly and respond to the user's operation. In FIG. 3A, the feedback processing unit 150 includes a trigger 152, and a pivot end 142 of the operating handle 140 is correspondingly connected to the trigger 152. The user can hold and push the operating handle 140 to generate a displacement of the pivot end 142. When the pivot end 142 of the operating handle 140 is displaced to output a mechanical energy to the trigger 152, the operating handle 140 can push the trigger 152 accordingly. Therefore, the harmonic scalpel 120 and the clamp 130 can be controlled by the operating handle 140 to generate a clamping action.

In addition, in FIG. 3B, the feedback processing unit 150 may include a trigger 152, a motor 154, and a connecting rod 156. Both ends of the connecting rod 156 are connected to the trigger 152 and the motor 154, respectively. The motor 154 may output a mechanical energy to the connecting rod 156 so that the connecting rod 156 drives the trigger 152 to generate a displacement. In addition, the motor 154 is controlled by the operating handle 140 so that the motor 154 can push the trigger 152. Therefore, the harmonic scalpel 120 and the clamp 130 can be controlled by the operating handle 140 to generate a clamping action.

That is to say, in FIG. 3B, an induction signal is transmitted between the operating handle 140 and the motor 154, for example, by wireless communication. When the operating handle 140 is forced to move, the feedback processing unit 150 can receive an induction signal to activate the motor 154 and push the trigger 152.

In addition, please refer to FIGS. 3A and 3B, the feedback processing unit 150 may include an indicator, such as LEDs 117 or other light sources, and the indicator includes at least one light signal for displaying the intensity of the indication signal. For example, the indicator indicates the grip feedback force of the clamp 130 or the grip feedback force of the operating handle 140 through the number of LEDs 117. The more the lights, the stronger the force; on the contrary, the fewer the lights, the weaker the force.

In another embodiment, the feedback processing unit 150 may include a speaker 118, such as a horn or other sound generating device, and the speaker 118 may generate at least one audio frequency to indicate the strength of the indication signal. For example, the speaker 118 indicates the clamping feedback force of the clamp 130 or the grip feedback force of the operating handle 140 through the level of the sound. The higher the sound, the stronger the force; on the contrary, the lower the sound, the weaker the force.

As can be seen from the above description, in the surgical tool clamping feedback device 100 of the present embodiment, the indicator 117 and/or the speaker 118 are installed on the machine body 110 to generate a light signal and/or an audio frequency to provide a visual and/or auditory feedback for the user to know the clamping feedback force of the clamp 130 or the grip feedback force of the operating handle 140 and respond to the user's operation. Therefore, the present invention can overcome the limitations of traditional minimally invasive surgery in term of visual and tactile aspects.

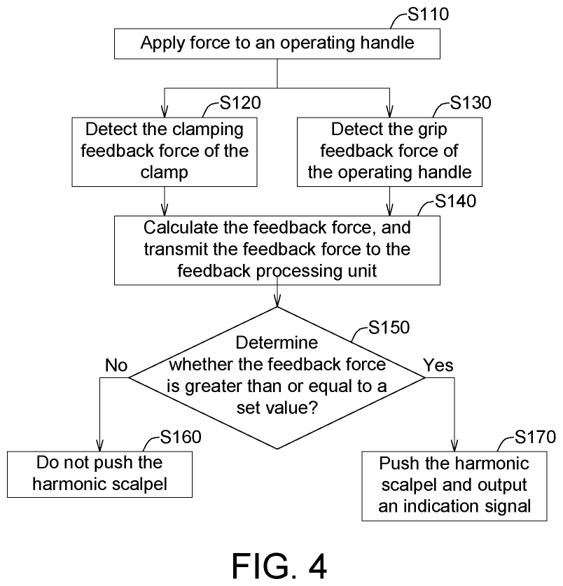

In addition, please refer to FIG. 4, which is a schematic flow chart of a surgical tool clamping feedback method according to an embodiment of the present invention. The following steps are implemented by the surgical tool clamping feedback device 100 shown in FIG. 1, for example, but not limited thereto, the surgical tool clamping feedback device 100 may include a surgical tool 101, a force feedback sensor 132 (144) and a feedback processing unit 150. In step S110, the user applies force to the operating handle 140 to perform a clamping action. In step S120, the clamping feedback force of the clamp 130 is detected according to the force feedback sensor 132 shown in FIGS. 2A and 2B, or in step S130, the grip feedback force of the operating handle 140 is detected according to the force feedback sensor 144 shown in FIGS. 3A and 3B, and the detected feedback force is configured to indicate the force of the surgical tool 101 on the organs and tissues. In step S140, the clamping or grip feedback force is calculated, and the clamping or grip feedback force is transmitted to the feedback processing unit 150, so that the feedback processing unit 150 can determine whether the surgical tool 101 clamps the tissue or blood vessel or not according to the feedback force. In step S150, the feedback processing unit 150 determines whether the feedback force is greater than or equal to a set value, and if it is less than the set value, step S160 is performed, and the harmonic scalpel 120 is not pushed. On the other hand, if it is greater than or equal to the set value, step S170 is performed to push the harmonic scalpel 120 and output an indication signal to respond to the user's operation. The indication signal represents the clamping feedback force of the clamp 130 or the grip feedback force of the operating handle 140 in a visual or auditory manner, so as to overcome the limitations of traditional minimally invasive surgery in term of vision and tactile aspects.

While the invention has been described by way of example and in terms of a preferred embodiment, it is to be understood that the invention is not limited thereto. On the contrary, it is intended to cover various modifications and similar arrangements and procedures, and the scope of the appended claims therefore should be accorded the broadest interpretation so as to encompass all such modifications and similar arrangements and procedures.

What is claimed is:

1. A surgical tool clamping feedback device, comprising:
a surgical tool comprising a machine body, a harmonic scalpel, a clamp and an operating handle, wherein the machine body is configured to install the harmonic scalpel, the harmonic scalpel is configured to install the clamp and output an ultrasonic wave to the clamp, the operating handle is movably disposed on the machine body for a user to hold and operate the clamp;
a force feedback sensor linked with the operating handle to detect a grip feedback force of the operating handle; and
a feedback processing unit configured to receive the grip feedback force of the operating handle and correspondingly output an instruction signal to the user.

2. The clamping feedback device according to claim 1, wherein the force feedback sensor is disposed in the machine body and linked with the operating handle.

3. The clamping feedback device according to claim 1, wherein the feedback processing unit comprises a trigger, a pivot end of the operating handle is correspondingly connected to the trigger, when the pivot end of the operating handle is displaced to output a mechanical energy to the trigger, the operating handle pushes the trigger.

4. The clamping feedback device according to claim 1, wherein the feedback processing unit comprises a trigger, a motor and a connecting rod, the connecting rod connects the trigger and the motor, and the motor outputs a mechanical energy to the connecting rod, so that the connecting rod drives the trigger to produce a displacement, and the motor is controlled by the operating handle to push the trigger.

5. The clamping feedback device according to claim 4, wherein a sensing signal is transmitted between the operating handle and the motor by wireless communication.

6. The clamping feedback device according to claim 1, wherein the machine body comprises a mounting portion and a handle portion, the mounting portion has a sleeve for mounting the harmonic scalpel, and the handle portion is located on a bottom of the mounting portion, and the handle portion is configured to operate in cooperation with the operating handle.

7. The clamping feedback device according to claim 6, wherein the handle portion is detachably disposed on the mounting portion, and the operating handle is pivotally disposed on the handle portion.

8. The clamping feedback device according to claim 6, wherein the operating handle is pivotally disposed on the mounting portion.

9. The clamping feedback device according to claim 1, wherein the feedback processing unit comprises an indicator, and the indicator generates at least one light signal to indicate a strength of the indication signal.

10. The clamping feedback device according to claim 1, wherein the feedback processing unit comprises a speaker, and the speaker generates at least one audio frequency to indicate a strength of the indication signal.

11. The clamping feedback device according to claim 1, wherein the force feedback sensor comprises a pressure sensor or a tension sensor.

12. A surgical tool clamping feedback method for a surgical operation, the surgical tool comprises a machine body, a harmonic scalpel, a clamp and an operating handle, wherein the machine body is configured to install the harmonic scalpel, the harmonic scalpel is configured to install the clamp and output an ultrasonic wave to the clamp, the operating handle is movably disposed on the machine body for a user to hold and operate the clamp, the method comprising:
apply force to the operating handle;
detecting a clamping feedback force of the clamp or detecting a grip feedback force of the operating handle; and
according to the clamping feedback force of the clamp or the grip feedback force of the operating handle, an indication signal is correspondingly output to the user.

13. The clamping feedback method according to claim 12, wherein a force feedback sensor is disposed in the harmonic scalpel, and the force feedback sensor is linked with the clamp to detect the clamping feedback force of the clamp.

14. The clamping feedback method according to claim 12, wherein a force feedback sensor is disposed in a jaw of the clamp, and the force feedback sensor is linked with the clamp to detect the clamping feedback force of the clamp.

15. The clamping feedback method according to claim 12, wherein a force feedback sensor is disposed in the machine body, and the force feedback sensor is linked with the operating handle to detect the clamping feedback force of the clamp.

16. The clamping feedback method according to claim 12, wherein the machine body comprises a trigger, a pivot end of the operating handle is correspondingly connected to the trigger, and when the pivot end of the operating handle is displaced to output a mechanical energy to the trigger, the operating handle pushes the trigger.

17. The clamping feedback method according to claim 12, wherein the machine body comprises a trigger, a motor and a connecting rod, the connecting rod connects the trigger and the motor, and the motor outputs a mechanical energy to the connecting rod, the connecting rod drives the trigger to generate a displacement, and the motor is controlled by the operating handle to push the trigger.

18. The clamping feedback method according to claim 17, wherein a sensing signal is transmitted between the operating handle and the motor by wireless communication.

19. The clamping feedback method according to claim 12, wherein the indication signal comprises at least one light signal to indicate a strength of the indication signal.

20. The clamping feedback method according to claim 12, wherein the indication signal comprises at least one audio frequency to indicate a strength of the indication signal.
